(19) United States
(12) Patent Application Publication
(10) Pub. No.: US 2009/0163778 A1
(43) Pub. Date: Jun. 25, 2009

(54) Personal Warning Apparatus

(76) Inventor: Paul Richard Sommerville, Glasgow (GB)

Correspondence Address: Gregory B. Coy;Krieg DeVault LLP, One Indiana Square, Suite 2800, Indianapolis, IN, 46204, US

(21) Appl. No.: 12/087,243

(22) PCT Filed: Dec. 28, 2006
(86) PCT No.: PCT/GB2006/004951

§ 371 Date: Nov. 17, 2008

(30) Foreign Application Priority Data
Dec. 28, 2005 (GB) 0526478.3
Oct. 28, 2006 (GB) 0621500.8

(51) Int. Cl. A61B 5/00

(52) U.S. Cl. 600/301

(57) ABSTRACT

Personal warning apparatus and method for use in particular with infants susceptible to Sudden Infant Death Syndrome, has a relative motion detector to be positioned on or about an individual, and a communication means operatively connected thereto optionally via an evaluation module, the apparatus featuring a predetermined activation threshold to mitigate false alarms.

The present invention relates to a personal warning apparatus. In particular, the present invention relates to an apparatus and method for detecting and preventing the onset of Sudden Infant Death Syndrome. The present invention also relates to an apparatus and method for detecting and preventing local environmental conditions associated with Sudden Infant Death Syndrome.

Sudden Infant Death Syndrome (SIDS) or “cot death” is known to primarily affect infants aged between 3 and 9 months. SIDS occurs unexpectedly and quickly to apparently healthy infants, usually during periods of sleep. There remains some debate as to the causative factors in SIDS, although it is established that ensuring that the infant's breathing remains unobstructed can help to reduce the risk. In addition, it has been proposed that preventing the infant from becoming too hot, and ensuring that the infant sleeps on its back, can help to prevent the risk of SIDS.

At present, several different types of apparatus and methods exist for detecting SIDS. However, the existing apparatus and methods have several disadvantages which restrict their effectiveness and ease of use. For example, many existing SIDS detection systems use detectors that sense movement of the infant. However, many of these detection systems are triggered by minor movements or when the infant stops moving for only a short period of time, creating many false alarms. Furthermore, several of these motion detectors comprise a single unit which is attached to the infant. These motion detection units have no fixed point of reference and are therefore more susceptible to false alarms.

Other existing apparatus for detecting SIDS measure the breathing pattern, the temperature, the heartbeat or other vital signs of the infant (some of these devices are known as apnea alarms). However, as infants' heartbeats and breathing patterns are often irregular, such devices can generate false alarms.

It can be seen that existing SIDS detection systems are prone to causing false alarms, and provide no way of preventing the initial onset of SIDS. It is of particular importance to develop a SIDS detection system that does not produce false alarms, as these can prove stressful and time consuming for the infant's carer. Moreover, numerous false alarms lessen the impact of the alarm on the carer, such that the carer is more likely to inadvertently neglect a genuine alarm. In addition several prior art systems, such as apnea alarms, detect SIDS rather than the causative factors. As such, these alarms provide no way of pre-empting or preventing SIDS. Therefore, it is an object of the present invention to overcome at least some of the drawbacks associated with the prior art.

A further object of the invention is to provide an apparatus and method for detecting the onset of SIDS.

A still further object of the invention is to provide an apparatus and method for preventing the onset of SIDS.

Further aims and objects of the invention will become apparent from a reading of the following description.

According to a first aspect of the present invention there is provided a personal warning apparatus comprising at least one relative motion detector adapted to be positioned on or around the body of an individual, an evaluation module, and at least one communication means, the at least one relative motion detector being operatively linked to the evaluation module, and the evaluation module being operatively linked to the at least one communication means, wherein the evaluation module responsive to an evaluation is able to send an alarm signal to the at least one communication means, and wherein the evaluation module has a predetermined activation threshold.

The predetermined activation threshold may be associated with the at least one relative motion detector, and optionally also a temperature sensor, and is selected to mitigate generation of false alarms.

Preferably the evaluation module is adapted to sense motion indicative of the onset of Sudden Infant Death Syndrome.

The evaluation module can be adapted to activate the at least one communication means on sensing conditions associated with the onset of Sudden Infant Death Syndrome.

The evaluation module can be a suitably programmed processor. The processor can be configured to evaluate detected motion.

Optionally the processor is a microprocessor.

Alternatively the processor is an application specific integrated circuit.

Preferably the apparatus further comprises a detector unit adapted to be positioned on or around the body of an individual.

Optionally, the apparatus further comprises an alarm unit remote from the detector unit, the detector unit being operatively connected to the alarm unit.

Preferably the apparatus further comprises a relay unit remote from the alarm unit and the detector unit, the detector unit being operatively connected to the relay unit, and the relay unit being operatively connected to the alarm unit.

Optionally the detector unit comprises the at least one relative motion detector, the evaluation module and at least one communication means.

Alternatively the detector unit comprises the at least one relative motion detector and at least one communication means, and the alarm unit comprises at least one communication means, the evaluation module and an alarm, the evaluation module being operatively connected to the alarm.

Preferably the detector unit comprises the at least one relative motion detector and at least one communication means, and the relay unit comprises at least one communication means and the evaluation module, and the alarm unit comprises at least one communication means and an alarm, the at least one communication means in the alarm unit being operatively connected to the alarm.

According to a second aspect of the present invention there is provided a personal warning apparatus comprising a device to be positioned on or around the body of an individual, the device comprising at least one relative motion detector operatively linked to at least one communication means, wherein the relative motion detector responsive to detection of relative motion is able to send an alarm signal to the at least one communication means, and wherein the at least one relative motion detector has a predetermined activation threshold.

Optionally the predetermined activation threshold is adjustable.

The relative motion detection means can be adapted to detect rotational movement. The rotational movement can be with respect to the longest axis of an individual's body. That is, with respect to the individual's height. Therefore, this can be used to monitor whether the infant has moved onto their side.

The relative motion detection means can be adapted to detect vertical movement. When an individual is lying down the vertical movement is with respect to the shortest axis of their body. Therefore, this can be used to monitor the individual's breathing.

The relative motion detection means can be adapted to detect lateral movement. The lateral movement is in the direction of the longest axis of the individual's body. That is with respect to the individual's height. Therefore, this can be used to monitor whether the infant has slid underneath any blankets or bed clothing.

The apparatus may further comprise at least one alert generating means, which can be an alarm.

The communication means and the alert generating means can be the same device which can be an audible and/or visible alarm. The alert generating means can be remote from the relative motion detection means. For example, an alarm can be with a carer who is in a different room from the infant.

Preferably the at least one communication means is a means for transmitting and receiving information wirelessly.

Preferably the means for transmitting and receiving information wirelessly is a radio frequency transceiver.

Preferably the at least one relative motion detector comprises an accelerometer.

The accelerometer can be a triaxial accelerometer.

The accelerometer can comprise a first biaxial accelerometer. Optionally the accelerometer comprises a first and a second biaxial accelerometer configured such that they detect movement in three dimensions.

Optionally, the accelerometer can comprise a first uniaxial accelerometer and a second uniaxial accelerometer configured such that they can detect movement in two dimensions. Alternatively, the accelerometer can comprise a first, a second and a third uniaxial accelerometer configured such that they can detect movement in three dimensions.

Preferably the accelerometer is a micro-machined accelerometer (MMA).

Optionally the at least one relative motion detector comprises a magnetic switch and an associated magnet.

The apparatus may further comprise a motion restriction means adapted to restrict the movement of an individual.

The motion restriction means may be a sleeping sack.

Optionally a magnetic switch is attached to the sleeping sack, and an associated magnet is adapted to be positioned on or around the body of an individual, or vice versa.

The sleeping sack may be configured to receive a removable conduit.

Preferably the apparatus further comprises a temperature sensor adapted to be positioned on or around the body of an individual.

According to a third aspect of the present invention there is provided a method of generating a personal warning comprising the steps of:
detecting the movement of an individual relative to at least one plane of the individual's body using at least one relative motion detector; and
activating a communication means based on the relative motion detected;
   wherein the communication means is activated when the relative motion of the individual is outwith a predetermined activation threshold.

The predetermined activation threshold may be adapted to mitigate generation of false alarms.

The method may comprise the further step of evaluating the relative motion detected using an evaluation module.

The method may comprise the further step of partially restricting the movement of the individual.

Optionally the method comprises the further step of monitoring the temperature of the individual.

Preferably the communication means is activated when the temperature of the individual deviates from a predetermined temperature range.

Optionally the communication means is an alarm signal.

The alarm signal may be activated at a location remote from the individual.

The present invention will now be described by way of example only, with reference to the accompanying drawings in which:

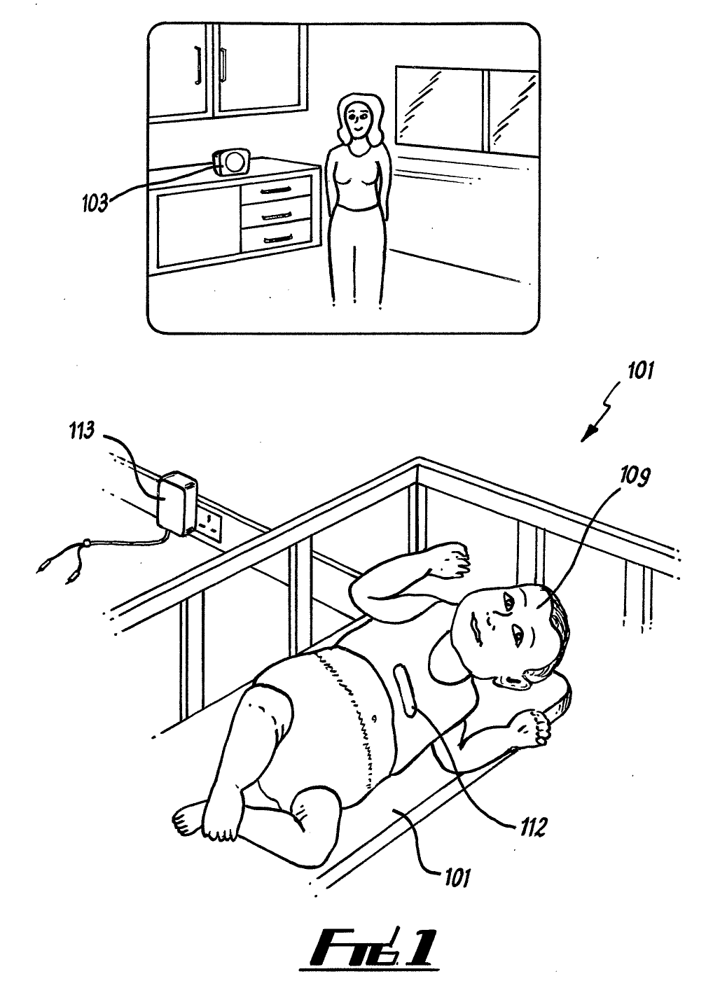

FIG. 1 is a representation of one embodiment of the SIDS alarm apparatus in accordance with the present invention; and

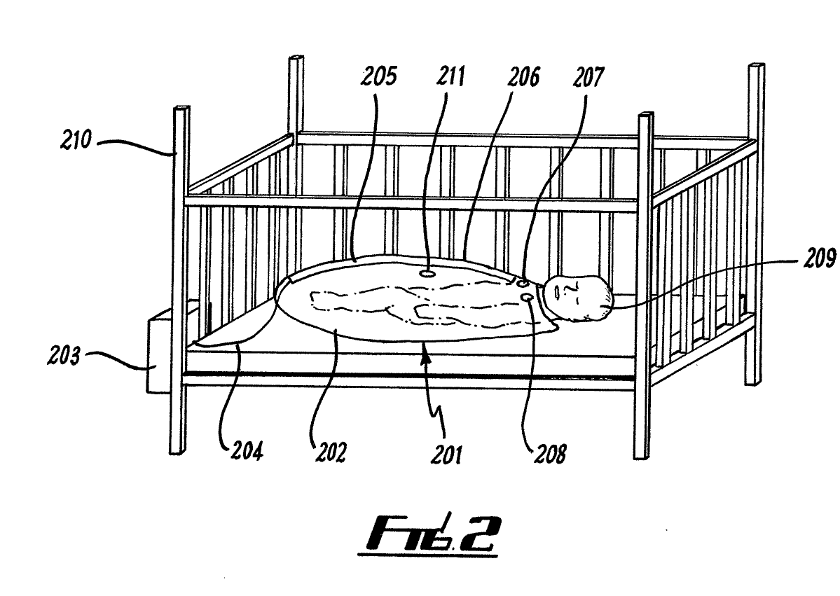

FIG. 2 is a representation of a further embodiment of the SIDS alarm apparatus containing a sleeping sack in accordance with the present invention.

Referring to FIG. 1 the SIDS prevention and detection apparatus is generally depicted at 101 and is comprised of a detector unit 112, a signal repeater or relay unit 113 and alarm unit 103. The detector unit 112 contains a radio-frequency transmitter microchip (not shown) and communicates with the relay unit 113 using radio frequency signals. The detector unit 112 contains a triaxial accelerometer (not shown) which measures rotational, vertical and lateral movement. The detector unit 112 is encased in a liquid resistant pouch (not shown). The pouch is secured to the chest of an infant 109, at rest in a cot 110. The detector unit 112 also contains a temperature sensor (not shown) to monitor the infant's temperature. The relay unit 113 contains a radio-frequency transceiver microchip (not shown) and communicates with the alarm unit 103, which contains a radio-frequency receiver (not shown), using radio-frequency signals. The relay unit also contains a microprocessor which acts as an evaluation module. The microprocessor evaluates the accelerometer readings and determines whether or not an alarm should be generated. If an accelerometer reading is evaluated as high risk, the microprocessor sends an alarm signal to the radio-frequency transceiver microchip which communicates with the alarm unit 103, which in turn generates an alarm.

The evaluation module can be a suitably programmed processor, microprocessor or application specific integrated circuit, configured to evaluate detected motion and/or temperature.

The evaluation module is responsive to the evaluation performed. If it concludes that a signal, or set of signals, is associated with risk to the individual it will send an alarm signal, by way of an electrical signal, to the radio-frequency transceiver. The radio frequency transceiver will transmit a signal to the alarm unit which will then raise an alarm. As the evaluation module is programmable and adjustable, it can be set to have predetermined movement and/or temperature thresholds, which mitigates the generation of false alarms. These thresholds are activation thresholds beyond which the evaluation module will conclude that the infant is at risk. In particular, the evaluation module can be programmed such that it activates the radio-frequency transceiver, and generates an alarm, on sensing movement and/or temperature conditions associated with the onset of SIDS.

In the example described, the components of the detector unit are linked by way of a printed circuit board and the detector unit is powered by a battery. The components of the relay unit are also linked using a printed circuit board, and the relay unit is mains electricity powered. Similarly, the components of the alarm unit are linked using a printed circuit board and the alarm unit is mains electricity powered. The detector unit, relay unit and alarm unit are linked by radio-frequency transmitters/receivers or transceivers and thus are operatively connected.

The evaluation module is adapted to be sensitive to motion indicative of the onset of Sudden Infant Death Syndrome. This can be motion associated with the infant adopting a high risk orientation or can be a motion pattern associated with cessation of breathing.

The detector unit can be removed from the pouch so that the pouch can be washed. The liquid resistant material prevents the ingress of liquid into the pouch and therefore protects the detector and transmitter equipment. In this example the pouch containing the detector unit is secured against the infant's chest using an adjustable strap. In an alternative embodiment the temperature sensor is located in the pouch.

The apparatus can contain one or more temperature sensors that measure one or both of the infant's temperature and the ambient temperature.

The detector can be secured against the infant's chest by any suitable means, including for example a harness or hook and loop fasteners. In one embodiment, the detector unit is placed in a pouch in a baby vest comprising wicking fabric, a padded chest area and hook and loop fastenings. The detector unit can be removed from the infant's clothing for washing.

In an alternative embodiment the detector unit is integral to the infant's clothing.

In use, the detector unit communicates the movements of the infant with the relay unit using small, low-powered digital radio transceivers. The same technology is used to communicate between the relay unit and the alarm unit.

In a further alternative embodiment, the detector unit communicates directly with the alarm unit, and there is no relay unit present. In this embodiment the detector unit also comprises the evaluation module which works in the normal way, sending an alarm signal to the alarm unit in the event of a high risk condition being assessed. Alternatively, the evaluation module is placed in the alarm unit rather then the detector unit. The detector unit is in constant communication with the alarm unit. If the evaluation module detects high risk condition an alarm is generated.

In a still further embodiment, the detector unit further comprises the evaluation module and an alarm, which acts as a communication means. In this particular embodiment it is not necessary for the detector unit to contain a radio-frequency transceiver, although it may optionally do so. The detector unit may act alone to detect, evaluate a high risk condition, and create an audible and/or visible alarm.

Accelerometer devices measure the acceleration and therefore movement in one or more directions. In this example one triaxial accelerometer capable of measuring in all three x, y and z axes is used. It is also feasible that two biaxial accelerometers both measuring in two axes could instead be used, as could three uniaxial accelerometers. Monitoring movement in one axis confirms that the infant is breathing. Detecting when the infant moves onto its side is possible by monitoring movement in another axis. Significant movement in the remaining axis detects when the infant moves up or down the cot, possibly under their blanket.

In the example described the accelerometer acts as a relative motion detector and the radio-frequency transmitters/receivers in the detector unit, the relay unit, and the alarm unit, act as communication means.

When the accelerometer detects conditions associated with the onset of SIDS, it sends an electrical signal to the microprocessor which acts as an evaluation module. If the evaluation module senses that the infant is at risk, it will send an electrical signal to the radio-frequency transmitter which in turn transmits a radio-frequency signal to the relay unit which acts as an intermediate transceiver unit. The relay unit transmits a radio-frequency signal to the alarm unit which acts as a remote alert generating means, and the alarm unit generates an audible and/or visible alarm to indicate to a carer that the infant is in a high risk state.

The accelerometer can detect rotational movement. The rotational movement is with respect to the longest axis of an infant's body. That is, with respect to the infant's height. Therefore, this can be used to monitor whether the infant has moved onto their side. In use, the rotational movement threshold can be set to between 30° and 45°.

One way of performing this measurement is to measure the acceleration readings on three separate axes to determine their orientation. As the accelerometers are effectively measuring an effect of gravity, an axis in line with the ground will give a reading of g. The same axis oriented at 45° will give a reading of 0.5 g. Thus, the orientation of the infant can be detected.

The accelerometer can also detect vertical movement. When an infant is lying down the vertical movement is with respect to the shortest axis of its body. This can be used to monitor the infant's breathing by, for example, averaging a number of acceleration readings (generated by the movement of the infant's chest, i.e. breathing) and determining if the readings have varied by a particular threshold amount. If the readings do not vary by this amount, the device detects that the infant has stopped breathing.

Furthermore the accelerometer can detect lateral movement. The lateral movement is in the direction of the longest axis of the infant's body. That is with respect to the infant's height. This detection requires the use of high accuracy, fast sampling and low noise. The accelerometer provides a measure of the acceleration at a particular sampling instant and thus can be used to indirectly measure distance moved in a particular direction. Therefore, this can be used to monitor whether the infant has slid underneath any blankets or bed clothing.

An advantage of the present invention is that the evaluation module used is adjusted to have predetermined activation thresholds, i.e., varying the sensitivity levels. The thresholds can be preset during manufacture in accordance to the average, safe movements of an infant during sleep. In an alternative embodiment the thresholds can be adjusted by the infant's carer in accordance with individual sleeping habits. This requires apparatus with which the carer can make the necessary adjustments. For example, using suitable software and an existing computer the thresholds can be adjusted and the infant monitored. The same software can be used to display the temperature of the infant and to raise an alarm.

Alternatively, the thresholds of the evaluation unit can be adjusted using dials or the like attached to the detector unit, the relay unit and/or the alarm unit. Such adjustments can be made manually and without the requirement for a personal computer.

This is particularly useful, for example, for avoiding false alarms when an infant enters deep sleep. In deep sleep, an infant's breathing is more likely to be irregular or shallow, and thus it is useful to adjust the sensitivity of the evaluation of the accelerometer that monitors breathing.

In use the accelerometer(s) detect movement in at least one plane of the infant's body, and communicate the measurements taken to a suitably programmed microprocessor, which evaluates the readings taken. If the microprocessor concludes that a reading corresponds to a high risk orientation or cessation of breathing it activates an alarm. The alarm can be activated via a radio-frequency transceiver or can be activated directly by the microprocessor. The microprocessor has preset and optionally programmable thresholds associated with the movement of the accelerometers. These thresholds are selected to mitigate the generation of false alarms.

Also in use, the movement of the infant may be partially restricted using, for example, a sleeping sack. In addition, the infant's temperature and/or ambient temperature may be monitored by temperature sensors and optionally evaluated by the microprocessor. When the temperature deviates from an acceptable range of values, or a high risk position or cessation of breathing is detected, an alarm or alarm signal is activated. The alarm signal may be remote from the device monitoring the infant.

FIG. 2 is a representation of a further embodiment of the SIDS alarm apparatus in accordance with the present invention.

Referring to FIG. 2 the SIDS prevention and detection apparatus is generally depicted at 201 and is comprised of a sack 202 and an alarm 203. The sack 202 is connected to the alarm 203 via wiring 204. Further wiring (not shown) is encased in a removable conduit 205 which slots into a channel 206 in the sack 202. The sack 202 comprises a magnetic switch 207 that is associated with a magnet 208 which is attached to an infant 209 at rest in a cot 210. The sack 202 further comprises a temperature sensor 211 for monitoring the infant's temperature. The temperature sensor 211 and the magnetic switch 207 are connected to the wiring (not shown) encased in the removable conduit 205.

The removable conduit 205 provides that all wiring for the alarm 203, the magnetic switch 207 and the temperature sensor 211 is isolated from the infant 209. This ensures the safety of the infant 209 and makes the sack 202 more comfortable for the infant 209 to sleep in. Consequently, the infant 209 is less restless during sleep. The removable conduit 205 is made from a liquid resistant material to prevent ingress of liquids. The removable conduit 205 can be separated from the sack 202, which is particularly useful for the purposes of washing the sack 202. In this example the removable conduit 205 attaches to the channel 206 in the sack 205 by way of a hook and loop fastening system.

In the example illustrated in FIG. 2, the magnetic switch and associated magnet act as a relative motion detector with an activation threshold that can be predetermined. This predetermined activation threshold can be preset or may be adjustable such that a user can set the threshold to their desired level.

In an alternative embodiment the wiring is integral to the sack and does not run in a removable channel. In this alternative embodiment, the wiring may still be removed from the sack for the purposes of washing or otherwise. The sack may itself provide a degree of water resistance.

In the embodiment shown in FIG. 2 the alarm 203 is connected to the sack 202 via wiring 204 and the sack 202 contains further wiring (not shown) for the temperature sensor 211 and the magnetic switch 207. The magnetic switch 207 is fixed in position, stitched into the lining of the sack 202.

In an alternative embodiment, the wiring is replaced by remote transceivers (such as radio frequency transmitters and radio frequency receivers) such that the temperature sensor and the magnetic switch can communicate wirelessly with the alarm.

Again referring to FIG. 2, the alarm 203 is an audible and visual alarm which is attached to the cot 210. In an alternative embodiment, the apparatus communicates with a remote device using means such as radio frequency communication, such that a remote alarm is triggered.

The remote alarm may be placed next to a carer who is remote from the infant.

Referring once more to FIG. 2, in use the infant 209 is fitted with a magnet 208 on its chest and is placed on its back in the sack 202. The sack 202 fits securely around the infant 209 such that minor movements are not constrained, but such that larger movements, for example turning in the sack 202 or sliding inside the sack 202, are prevented. This affords the infant 209 a more comfortable and safer sleeping environment.

The magnetic switch 207 is adjusted to a predetermined activation threshold such that if the infant 209 moves as part of the normal sleeping process the magnet 208 and magnetic switch 207 remain associated and the magnetic switch 207 is not triggered. However, should the sack 202 fail to constrain the infant 209 and allow the infant 209 to move into a “high risk” position (i.e. if the infant 209 slides inside the sack 202 or turns onto its front), the association between the magnet 208 and the magnetic switch 207 is broken, triggering the alarm 203. In this embodiment the predetermined activation threshold can be adjusted. In an alternative embodiment, the predetermined activation threshold is not adjustable.

Once more referring to FIG. 2, the magnetic switch 207 and magnet 208 remain associated when they stay within a certain distance of each other as set by the predetermined activation threshold. That is, the two devices will tolerate a certain degree of relative movement without becoming dissociated. As long as the magnetic switch 207 and the magnet 208 are associated, the magnetic switch 207 will not be activated and the alarm 203 will not be triggered. This affords the present apparatus a degree of flexibility in that the infant 209 may make “safe” movements without triggering the alarm 203. Therefore, the present apparatus prevents false alarms from occurring.

In the example described, the magnetic switch and the magnet act as a relative motion sensor that has a degree of tolerance to minor movements.

Referring still to FIG. 2, in use the temperature sensor 211 affixed to the sack 202 continuously monitors the temperature of the infant 209. If the temperature deviates from the normal accepted temperature range, the alarm 203 is triggered. The alarm 203 may be set to produce a different type of alert depending on whether the temperature sensor 211 or the magnetic switch 207 triggers the alarm 203. For example, the alarm 203 may be an audible alert of different pitch for the temperature sensor 211 and the magnetic switch 207.

In a further embodiment the temperature sensor is attached to the infant's abdomen. If the infant's temperature deviates out with the normal accepted temperature range, the temperature sensor will trigger the alarm. The temperature sensor can be adapted to be positioned on or around the infant or their clothing.

In a still further embodiment, the sleeping sack as described in reference to FIG. 2 may is used with the embodiments as described with reference to FIG. 1.

An advantage of the present invention is that it acts as a preventative safety measure in that the sack that restricts the infant's movement deters the infant from taking up a high risk orientation. In particular, the sack restricts the infant from turning onto its front and from sliding inside the sack. In these positions, the infant can re-inhale its own exhaled air, a proposed causative factor in SIDS.

A further advantage of the present invention is that it detects any movement by an infant into a high risk position. As such, the alarm is triggered before the infant stops breathing and before SIDS occurs. That is to say, the invention detects high risk environmental conditions associated with the onset of SIDS (by means of both the movement and the temperature sensor) rather than SIDS. Therefore, the present invention pre-empts SIDS and can allow a carer to attend to an infant in a high risk environment, before the infant stops breathing, increasing the chances of preventing SIDS.

Prior art SIDS alarms serve only to inform a carer that SIDS has occurred. As such, they do not afford the carer the opportunity to intervene, so that they might prevent SIDS from occurring. Furthermore, existing SIDS alarms do not combine a SIDS warning system with an apparatus for preventing an infant from adopting an orientation associated with a higher risk of SIDS. In addition, existing SIDS alarms are prone to false alarms, which detract from their impact in the event of a genuine SIDS incident. The SIDS alarm of the present invention has further applications, such as monitoring the breathing patterns, movement and/or temperature of the elderly, the infirm, or any individual susceptible to cessation of breathing, adopting a high risk orientation or experiencing abnormal temperature.

The apparatus of the present invention measures movement of the infant relative to a substantially fixed point. For example, the accelerometers measure relative to preset fixed planes. Alternatively the magnet is attached to the infant and the magnetic switch is attached to the sack. Due to the nature of the accelerometers and the evaluation module, and the nature of the interaction between the magnetic switch and the magnet, the apparatus of the present invention minimises the chances of false alarms occurring. That is, both the evaluation module and the magnetic switch have a activation threshold and will not be triggered by small “safe” movements by the infant. However, should the infant move into a “high risk” position, the evaluation module concludes from the accelerometer readings that a high risk position has been adopted, or the association between the magnetic switch and the magnet is broken, and the alarm is activated.

Improvements and modifications may be incorporated herein without deviating from the scope of the invention.

1. A personal warning apparatus comprising at least one relative motion detector adapted to be positioned on or around the body of an individual, an evaluation module, and at least one communication means, the at least one relative motion detector being operatively linked to the evaluation module, and the evaluation module being operatively linked to the at least one communication means, wherein the evaluation module responsive to an evaluation is able to send an alarm signal to the at least one communication means, and wherein the evaluation module has a predetermined activation threshold.

2. An apparatus as claimed in claim 1, wherein the evaluation module is adapted to sense motion indicative of the onset of Sudden Infant Death Syndrome.

3. An apparatus as claimed in claims 1 or 2, wherein the evaluation module is adapted to activate the at least one communication means on sensing conditions associated with the onset of Sudden Infant Death Syndrome.

4. An apparatus as claimed in any preceding claim, wherein the evaluation module is a suitably programmed processor.

5. An apparatus as claimed in claim 4, wherein the processor is configured to evaluate detected motion.

6. An apparatus as claimed in claims 4 or 5, wherein the processor is a microprocessor or application specific integrated circuit.

7. An apparatus as claimed in any preceding claim wherein the apparatus further comprises a detector unit adapted to be positioned on or around the body of an individual.

8. An apparatus as claimed in any preceding claim wherein the apparatus further comprises an alarm unit remote from the detector unit, the detector unit being operatively connected to the alarm unit.

9. An apparatus as claimed in any preceding claim wherein the apparatus further comprises a relay unit remote from the alarm unit and the detector unit, the detector unit being operatively connected to the relay unit, and the relay unit being operatively connected to the alarm unit.

10. An apparatus as claimed in claim 7 wherein the detector unit comprises the at least one relative motion detector, the evaluation module and at least one communication means.

11. An apparatus as claimed in claim 8 wherein the detector unit comprises the at least one relative motion detector and at least one communication means, and the alarm unit comprises at least one communication means, the evaluation module and an alarm, the evaluation module being operatively connected to the alarm.

12. An apparatus as claimed in claim 9 wherein the detector unit comprises the at least one relative motion detector and at least one communication means, and the relay unit comprises at least one communication means and the evaluation module, and the alarm unit comprises at least one communication means and an alarm, the at least one communication means in the alarm unit being operatively connected to the alarm.

13. A personal warning apparatus comprising a device to be positioned on or around the body of an individual, the device comprising at least one relative motion detector operatively linked to at least one communication means, wherein the relative motion detector responsive to detection of relative motion is able to send an alarm signal to the at least one communication means, and wherein the at least one relative motion detector has a predetermined activation threshold.

14. An apparatus as claimed in any preceding claim, wherein the predetermined activation threshold is adjustable.

15. An apparatus as claimed in any preceding claim, wherein the at least one relative motion detector is adapted to detect rotational movement with respect to the longest axis of the individuals body.

16. An apparatus as claimed in any preceding claim, wherein the relative motion detector is adapted to detect vertical movement with respect to the shortest axis of the individual's body.

17. An apparatus as claimed in any preceding claim, wherein the relative motion detector is adapted to detect lateral movement where lateral movement is in the direction of the longest axis of the individual's body.

18. An apparatus as claimed in claim 10 wherein the at least one communication means is an alarm.

19. An apparatus as claimed in claims 1 to 17, wherein the at least one communication means is a means for transmitting and receiving information wirelessly.

20. An apparatus as claimed in claim 19, wherein the means for transmitting and receiving information wirelessly is a radio frequency transceiver.

21. An apparatus as claimed any preceding claim, wherein the at least one relative motion detector comprises an accelerometer.

22. An apparatus as claimed in claim 21, wherein the accelerometer comprises a triaxial accelerometer.

23. An apparatus as claimed in claim 21, wherein the accelerometer comprises a first biaxial accelerometer.

24. An apparatus as claimed in claim 21, wherein the accelerometer comprises a first biaxial accelerometer and a second biaxial accelerometer, the first and second biaxial accelerometers configured such that they detect movement in three dimensions.

25. An apparatus as claimed in claim 21, wherein the accelerometer comprises a first uniaxial accelerometer and a second uniaxial accelerometer configured such that they can detect movement in two different dimensions.

26. An apparatus as claimed in claim 21, wherein the accelerometer comprises a first, a second and a third uniaxial accelerometer configured such that they can detect movement in three different dimensions.

27. An apparatus as claimed in claims 21 to 26, wherein the accelerometer comprises a micro-machined accelerometer (MMA).

28. An apparatus as claimed in claims 1 to 20, wherein the at least one relative motion detector comprises a magnetic switch and an associated magnet.

29. An apparatus as claimed in any preceding claim, wherein the apparatus further comprises a motion restriction means adapted to restrict the movement of an individual.

30. An apparatus as claimed in claim 29, wherein the motion restriction means is a sleeping sack.

31. An apparatus as claimed in claim 30, wherein a magnetic switch is attached to the sleeping sack, and an associated magnet is adapted to be positioned on or around the body of an individual, or vice versa.

32. An apparatus as claimed in claims 30 or 31, wherein the sleeping sack is configured to receive a removable conduit.

33. An apparatus as claimed in any preceding claim, wherein the apparatus further comprises a temperature sensor adapted to be positioned on or around the body of an individual.

34. A method of generating a personal warning comprising the steps of:
detecting the movement of an individual relative to at least one plane of the individual's body using at least one relative motion detector; and
activating a communication means based on the relative motion detected;
wherein the communication means is activated when the relative motion of the individual is outwith a predetermined activation threshold.

35. A method as described in claim 34 comprising the further step of evaluating the relative motion detected using an evaluation module.

36. A method as described in claims 34 or 35, wherein the method comprises the further step of partially restricting the movement of the individual.

37. A method as described in claims 34 to 36, the method comprising the further step of monitoring the temperature of the individual.

38. A method as described in claim 37, wherein the communication means is activated when the temperature of the individual deviates from a predetermined temperature range.

39. A method as described in claims 34 to 38, wherein the communication means is an alarm signal.

40. A method as described in claim 39, wherein the alarm signal is activated at a location remote from the individual.
